(19) United States
(12) Patent Application Publication
(10) Pub. No.: US 2017/0071975 A1
(43) Pub. Date: Mar. 16, 2017

(54) COMPOSITIONS AND METHODS FOR TREATING DEPRESSIVE DISORDERS

(71) Applicant: Steerwasher, LLC, St. Louis, MO (US)

(72) Inventor: Peter NAGELE, St. Louis, MO (US)

(21) Appl. No.: 15/308,921

(22) PCT Filed: May 12, 2015
(86) PCT No.: PCT/US15/30367

§ 371 Date: Nov. 4, 2016

Related U.S. Application Data
(60) Provisional application No. 61/991,993, filed on May 12, 2014.

(51) Int. Cl. A61K 33/00; A61K 45/06

(52) U.S. Cl. CPC A61K 33/00; A61K 45/06

(57) ABSTRACT

Compositions and methods for treating depressive disorders are disclosed. They are designed to provide effective and rapidly acting treatment for depression.

### CROSS REFERENCE TO RELATED APPLICATIONS

This application claims the benefit of U.S. Provisional Applications No. 61/991,993 filed May 12, 2014, the contents of which are hereby incorporated by reference.

### BACKGROUND

Field

The present disclosure relates to compositions and methods for treating depression, more specifically to the use of nitrous oxide for treating patients with treatment-resistant depression.

Related Art

Treatment-resistant depression is a particularly severe form of major depressive disorder. Affecting one in three patients with major depressive disorder (estimated prevalence in the United States is 10 million adults), patients with treatment-resistant depression often fail multiple treatments with standard antidepressants and have an unfavorable long-term prognosis. Therapeutic options for treatment-resistant depression are scarce.

Recent evidence, however, shows that a small, sub-anesthetic dose of ketamine—a general anesthetic—may provide a rapid and sustained antidepressant effect in patients with treatment-resistant depression. Unfortunately, ketamine has several unwanted side effects that restrict its clinical use. Being chemically related to phencyclidine (PCP, “angel dust”), ketamine causes hallucinations and illusions, has addictive properties, induces sedation, and must be administered intravenously by a physician experienced in sedation and anesthesia.

It may be appear somewhat unorthodox considering laughing gas (nitrous oxide) for treatment-resistant depression, but there is a strong biological rationale supporting this notion, because ketamine and nitrous oxide share their molecular mechanism of action. Like ketamine, nitrous oxide acts in the central nervous system predominantly by inhibiting NMDA receptors. NMDA receptor signaling has strongly been implicated in the neurobiology of depression. Given the similar mechanism of action, we therefore hypothesized that nitrous oxide may be an effective and rapidly acting treatment for treatment-resistant major depression

Thus, there remains an unmet need for treatments for depression that is only partially responsive to medication and intractable (e.g. ‘treatment-resistant’) depression.

### SUMMARY

Accordingly, disclosed herein are compositions and methods for treating depression. The methods are designed to provide rapid and marked antidepressant effects in patients with treatment-resistant depression.

Provided is a method of treating a depressive disorder in a subject in need thereof, comprising administering to the subject an effective amount of an inhaled gas comprising nitrous oxide.

Provided is an inhaled gas comprising nitrous oxide for use in treating a depressive disorder in a subject in need thereof.

Provided is the use of an inhaled gas comprising nitrous oxide for the manufacture of a medicament to treat a depressive disorder in a subject in need thereof.

### BRIEF DESCRIPTION OF THE DRAWINGS

FIG. 1 shows the effects of nitrous oxide treatment on depressive symptoms measured on the 21-point Hamilton Depression Rating Scale (HRDS). Patients were measured at three time points: baseline, 2 hours, and 24 hours after treatment completion. HDRS scores >18 indicate severe depression. A reduction of more than 50% on the HDRS scale is considered a treatment response. Nitrous oxide provided a significantly more pronounced reduction in depressive symptoms compared to placebo treatment (p=0.002). Of note, patients in antidepressant trials often respond to placebo treatment. Blue circles=nitrous oxide; black squares=placebo treatment. Symbols indicate mean±95% CI;

FIGS. 2A and 2B show clinical outcomes after nitrous oxide and placebo treatment;

FIG. 2A shows Rates of Response (defined as a reduction in Hamilton Depression Rating Scale >50%); and

FIG. 2B shows Rates of Remission (defined as complete resolution of depressive symptoms) 24 hours after treatment are shown. Compared to placebo, nitrous oxide had a 4-fold higher response (odds ratio 4.0, 95% CI 0.45-35.79) and 3-fold higher remission rate (OR 3.0, 95% CI 0.31-28.8); and

FIGS. 3A and 3B show effects of nitrous oxide treatment on depressive symptoms for only the first treatment session (10 patients each) measured on the 21-point Hamilton Depression Rating Scale (HRDS).

FIG. 3A shows the absolute changes on the HRDS compared to baseline, 2 hours, 24 hours and 1 week after treatment;

FIG. 3B shows the relative changes on the HRDS compared to baseline, 2 hours, 24 hours and 1 week after treatment. Nitrous oxide provides a significantly stronger reduction in depressive symptoms compared to placebo. HRDS scores at 1 week were derived when patients returned for their second session (=baseline HRDS score for session 2). 1 week HRDS scores after nitrous oxide treatment are significantly lower than at baseline, indicative of a sustained treatment effect;

FIG. 4 shows a cell plot (heat map) of individual responses of first treatment session measured on the Hamilton Depression Rating Scale (HRDS) shaded to indicate severity of symptoms (red—severe, blue—less severe). Each row represents an individual patient; patients in both cohorts (nitrous oxide and placebo) are not identical, i.e. this plot does not represent paired data; and

FIG. 5 shows the relative change adjusted for baseline.

### DETAILED DESCRIPTION

### Abbreviations and Definitions

To facilitate understanding of the disclosure, a number of terms and abbreviations as used herein are defined below as follows:

When introducing elements of the present disclosure or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

The term “about,” as used herein when referring to a measurable value such as an amount of a compound, dose, time, temperature, and the like, is meant to encompass variations of 20%, 10%, 5%, 1%, 0.5%, or even 0.1% of the specified amount.

The terms “depression” or “depressive disorder” as used herein refers to any nervous system disorder and/or mental condition characterized by, but not limited to, the following symptoms: depressed mood, anhedonia, feelings of intense sadness and despair, mental slowing, loss of concentration, pessimistic worry, agitation, self-deprecation, disturbed sleep patterns (e.g. insomnia, loss of REM sleep, or hypersomnia), anorexia, changes in appetite and weight loss or weight gain, Psychomotor agitation, decreased energy, decreased libido, and changes in hormonal circadian rhythms, withdrawal, altered daily rhythms of mood, activity, temperature and neuroendocrine function, and combinations thereof. Non-limiting examples of “depression” include major depressive disorder, bipolar depressed mood disorder, adjustment mood disorder, and post-partum mood disorder.

The terms “treatment,” “treating” or “treat” as used herein when referring to a condition, and as understood in the art, are defined to mean an approach for obtaining beneficial or desired results, including clinical results. Beneficial or desired clinical results can include, but are not limited to, alleviation of one or more symptoms of the condition, diminishment of extent of disease or condition, stabilized (i.e. not worsening) state of disease or condition, preventing spread of disease, delay or slowing of disease progression, palliation of the disease state, and remission (whether partial or total), whether detectable or undetectable.

The terms “subject” or “patient” as used herein are used interchangeably and mean all members of the animal kingdom (e.g. humans).

The term “subject in need of” as used herein when referring to nitrous oxide administration, means a subject having a condition that can be treated with nitrous oxide.

The term “effective amount” or “pharmaceutically effective amount” as used herein are used interchangeably, and are defined to mean the amount or quantity of nitrous oxide, which is sufficient to elicit an appreciable biological response when administered to a patient. It will be appreciated that the precise therapeutic dose will depend on the age and condition of the patient and the nature of the condition to be treated and will be at the ultimate discretion of the attendant physician.

### Compositions

One aspect of the present disclosure provides an inhaled gas comprising nitrous oxide for treating a depressive disorder in a subject in need thereof.

In certain embodiments, the inhaled gas comprises no more than 75% nitrous oxide by weight.

In certain embodiments, the inhaled gas comprises from about 5% to about 70% nitrous oxide by weight.

In certain embodiments, the inhaled gas comprises from about 20% to about 50% nitrous oxide by weight.

In various embodiments, the inhaled gas further comprises oxygen, nitrogen, xenon, or combinations thereof.

In certain embodiments, the inhaled gas comprises no more than 75% oxygen by weight.

In certain embodiments, the inhaled gas comprises from about 25% to about 75% oxygen by weight.

In certain embodiments, the inhaled gas comprises no more than 75% nitrogen by weight.

### Methods

Another aspect of the present disclosure provides new methods of treating a depressive disorder in a subject in need thereof, comprising administering to the subject an effective amount of an inhaled gas comprising nitrous oxide.

In certain embodiments, the depressive disorder is atypical depression, bipolar disorder, catatonic depression, depressive disorder not otherwise specified, depressive personality disorder, double depression, dysthymia, major depressive disorder, melancholic depression, minor depressive disorder, postpartum depression, post-traumatic stress disorder, psychotic major depression, recurrent brief depression, seasonal affective disorder, suicidality/acute suicide risk, or treatment-resistant major depression.

In particular embodiments, the depressive disorder is treatment-resistant major depression.

In certain embodiments, the subject is human.

### Dosage and Administration

In accordance with yet another aspect of the invention, the amount of nitrous oxide administered is sub-anesthetic.

In certain aspects, the inhaled gas is administered at a flow rate of about 0.1 liters to about 10.0 liters per minute. In certain embodiments, the inhaled gas is administered at a flow rate of about 0.5 liters to about 8.0 liters per minute. In particular an embodiment, the inhaled gas is administered at a flow rate of about 1.0 liters to about 2.0 liters per minute.

In certain aspects, the inhaled gas is administered for about 1 to 90 minutes. In certain embodiments, the inhaled gas is administered for about 30 to 60 minutes.

In certain aspects, the inhaled gas is administered at least one day every seven days of treatment. The inhaled gas may also be given every day, or every other day, or every third day, or every fourth day, or every fifth day, or every sixth day of a treatment period.

### Combinations and Combination Therapy

The present method can be used alone or in combination with other pharmaceutically active compounds, to treat conditions such as those previously described herein above. The inhaled gases of the present disclosure and other pharmaceutically active compound(s) can be administered simultaneously or sequentially. Accordingly, in one embodiment, the present invention comprises methods for treating a depressive disorder by administering to the subject an effective amount of an inhaled gas comprising nitrous oxide and one or more additional pharmaceutically active compounds.

In certain embodiments, the additional therapeutic agent is a selective serotonin reuptake inhibitor, serotonin-norepinephrine reuptake inhibitor, tricyclic antidepressant, or monoamine oxidase inhibitor. In particular embodiments, the additional therapeutic agent is Amitriptyline, Bupropion, Citalopram, Desvenlafaxine, Duloxetine, Escitalopram, Fluoxetine, Mirtazapine, Nortriptyline, Paroxetine, Sertraline, Trazodone, or Venlafaxine.

In another aspect, the present method can be used alone or in combination with other non-chemical means for treating a depressive disorder. In particular embodiments, the additional treatment comprises electroconvulsive therapy. In other embodiments, the additional treatment comprises psychotherapy.

### Vitamin B₁₂

A safety concern relates to nitrous oxide's inactivation of vitamin B₁₂. While a single exposure is very unlikely to result in clinically relevant hematological or neurological complications, the risk for such complications is substantially higher when nitrous oxide administrations are repeated within short periods. Hematological and neurological complications have mostly been reported among chronic nitrous oxide abusers or patients with chronic disturbances of folate metabolism. It is likely that for sustained antidepressant effect, nitrous oxide must be administered several times, which would increase the risk for such complications.

The inactivation of Vitamin B₁₂ and cobalamin-dependent enzymes, such as methionine synthase, is chemically irreversible and can last clinically up to one week until new enzyme is synthesized. In patients, the inactivating effects of N₂O on Vitamin B₁₂ can be observed by an increase in plasma total homocysteine because methionine synthase, the enzyme responsible for the conversion of homocysteine to methionine, requires Vitamin B₁₂ as co-enzyme.

Assays for measuring serum homocysteine levels are well known in the art, and have been recently reviewed—see for example “Performance characteristics of six homocysteine assays.” Am J Clin Pathol. 2008 December; 130(6):969-75.

### Homocysteine Monitoring

Thus, in accordance with yet another aspect of the disclosure, the method further comprises a step of monitoring the serum homocysteine concentration in the subject receiving treatment. In certain embodiments, the method includes measuring the serum homocysteine concentration in the subject; and
decreasing the amount of nitrous oxide administered when the measured serum homocysteine concentration in the subject is greater than about 15 μmol/L, and maintaining the amount of nitrous oxide administered when the measured serum homocysteine concentration in the subject is between about 5 μmol/L and about 14 μmol/L.

In certain embodiments, the serum homocysteine concentration is measured up to about six hours after administering the nitrous oxide.

In particular embodiments, the administering and measuring steps are repeated until the serum homocysteine concentration in the subject is between about 5 μmol/L and about 14 μmol/L.

### Co-Administration of Vitamin B

Thus, in accordance with yet another aspect of the disclosure, the method further comprises a step of sequential or co-administration of an additional agent for the treatment and/or prevention of a functional Vitamin B₁₂ deficiency. In certain embodiments, the additional therapeutic agent is Vitamin B₁₂, Vitamin B₆, Vitamin B₂, folic acid, methionine, cyanocobalamin, hydroxocobalamin, methylcobalamin, adenosylcobalamin, or combinations thereof.

Any appropriate mode of administration may be used, such as (but not limited to) oral, sublingual, intravenous and parenteral administration.

### Example

### Methods

In a blinded, randomized placebo-controlled crossover trial, 20 evaluable patients with treatment-resistant depression received either a treatment with 50% nitrous oxide/50% oxygen or 50% nitrogen/50% oxygen for one hour in random order. Primary endpoint was the change on the 21-point Hamilton Depression Rating Scale (HRDS).

### Study Design and Oversight

This study was designed as a randomized, placebo-controlled crossover pilot clinical trial. Patients had two treatment sessions that were 1 week apart (nitrous oxide or placebo). The order of the session was randomly assigned by a random number generator. Other than the gas mixture administered, both sessions were indistinguishable from each other in setting, setup, and monitoring.

### Blinding of the Study Treatment

First, both locations for treatment and psychiatric evaluation were physically separated from each other and no team member was allowed to enter the other space while a study patient was present. Second, records for the nitrous oxide treatment administration were kept separate from the case report forms until completion of the study. Third, all equipment used to provide the treatment was identical between nitrous oxide and placebo sessions.

The investigators were responsible for all aspects of the trial including design, protocol, data collection and analyses. The study was conducted in accordance to the protocol. There was no extramural support for this project. A data and safety monitoring board monitored the trial. The authors wrote the manuscript and vouch for the accuracy and completeness of the data and for the analysis. The study was approved by the Washington University in St. Louis institutional review board, and all patients provided written, informed consent.

### Patients

Patients were recruited from an existing database of treatment-resistant depression patients administered by the Washington University Department of Psychiatry as well as from the “Volunteers for Health” patient pool within the Washington University School of Medicine. Patients were eligible if (1) 18-65 years of age; (2) meeting the DSM-IV-TR criteria for major depressive disorder without psychosis, as determined using a structured clinical interview (the Mini International Neuropsychiatric Interview [MINI]); (3) a Hamilton Depression Rating Scale-21 item (HDRS-21) pre-treatment baseline score >18; (4) meeting criteria for treatment-resistant depression, defined as having had at least two adequate dose-duration, antidepressant medication failures in the current depressive episode and a lifetime failure of at least three antidepressant medication trials. Patients were excluded if (1) a history of bipolar disorder, schizophrenia, schizoaffective disorder, obsessive-compulsive disorder, panic disorder, or documented Axis II diagnoses; (2) active or recent substance abuse or dependence (“recent” defined as within the past 12 months; exception was made for nicotine use disorder); (3) the presence of acute medical illness that could interfere with study participation, including, but not limited to, significant pulmonary disease; (4) active suicidal intention; (5) active psychosis; (6) previous administration of NMDA-receptor antagonists (such as ketamine); (7) ongoing electroconvulsive therapy (ECT) treatment; (8) pregnant or breastfeeding women; (9) contraindications against the use of nitrous oxide: pneumothorax, middle ear occlusion, elevated intracranial pressure, chronic cobalamin and/or folate deficiency treated with folic acid or vitamin B₁₂. Patients were instructed not to suspend their standard of care treatment for major depression. Patients were required to maintain a stable medication regiment without changes for 4 weeks prior to initiation of the study and continue on the same dose throughout.

Between November 2012 and February 2014, we enrolled 24 patients with TRD into the trial. After excluding four patients (3 screen failures, 1 withdrawal), 20 patients were randomly assigned to a study group and completed the follow-up assessment. Patients had on average 19 years of depression history, failed 8 antidepressant drug treatments, and took two antidepressants at time of study participation (Table 1). The median score on the Hamilton Depression Rating Scale at enrollment was 23.5, indicative of very severe depressive symptoms (IQR 22.3-25.0).

TABLE 1
Baseline Characteristics

| | | |
|---|---|---|
| Age - yrs | 48 | [30-55] |
| Female Sex - no. (%) | 12 | (60%) |
| Race - no. (%) | | |
| White | 20 | (100) |
| Depression history - yrs | 19 | [11-27] |
| Number of failed treatments | 8 | [4-12] |
| Vagus nerve stimulator - no. (%) | 3 | (15) |
| History of ECT - no. (%) | 4 | (20) |
| Number of current antidepressant medications | 2 | [0-2] |
| History of migraine - no. (%) | 10 | (50) |
| History of hypothyroidism - no. (%) | 4 | (20) |
| Current medication - no. (%) | | |
| Antidepressants | 13 | (65) |
| SSRI | 6 | (30) |
| SNRI | 5 | (25) |
| Bupropion | 5 | (25) |
| Clomipramine | 1 | (5) |
| Atypical Antipsychotics | | |
| Quetiapine | 2 | (10) |
| Aripiprazole | 1 | (10) |
| Anticonvulsants | | |
| Lamotrigine | 3 | (15) |
| Other | | |
| Benzodiazepine | 2 | (10) |
| Atomoxetine | 1 | (5) |
| Dextroamphetamine | 1 | (5) |
| Methylphenidate | 1 | (5) |

### Treatment

Patients received either up to 50% nitrous oxide/50% oxygen (“treatment”) or 50% nitrogen/50% oxygen for 1 hour (“placebo”). The gas mix was administered via a standard anesthesia facemask through tubing connected to an anesthesia machine. A small sample connector line was inserted into the facemask allowing the measurement of inhaled and exhaled gas concentrations. Total gas flow was between 4-8 L/min. Patients were monitored during and after the treatment according to American Society of Anesthesiologists standard which includes continuous 3-lead ECG, pulsoximetry, non-invasive blood pressure and end-tidal CO₂ under the supervision of an attending-level anesthesiologists. After the one-hour treatment session, patients were transferred and monitored for 2 hours. A study team physician determined if the patients met criteria for discharge before patients were discharged.

Fifteen patients completed the full 60-minute treatment with nitrous oxide; in five patients the treatment either had to be interrupted or discontinued (emotional discomfort; regurgitation; claustrophobia; nausea and vomiting, see Table 2 for adverse events). The mean duration of nitrous oxide treatment was 55.6±2.5 (SD) minutes at an average inspiratory nitrous oxide concentration of 44% (37-45%, median, IQR). All patients completed the full 60-minute placebo treatment.

TABLE 2
Adverse Outcomes

| Adverse Event | Nitrous Oxide | | Placebo |
|---|---|---|---|
| Nausea and vomiting - no. (%) | 3 | (15%) | 0 |
| Headache - no. (%) | 2 | (10%) | 2 (10%) |
| Dizziness/Lightheadedness - no. (%) | 1 | (5%) | 2 (10%) |
| Numbness/Paresthesia | 2 | (10%) | 0 |
| Anxiety | 2 | (10%) | 0 |
| Panic Attack | 1 | (5%) | 0 |
| Hypercapnia | 0 | | 1 (5%) |
| Claustrophobia - no. (%) | 1 | (5%) | 0 |
| Hyperventilation - no. (%) | 1 | (5%) | 0 |
| Regurgitation - no. (%) | 1 | (5%) | 0 |

### Outcomes

Outcomes were assessed at six time points for each patient (three per session; two sessions): within each session at baseline, 2 hours, and 24 hours after treatment. Primary study endpoint was the change on the 21-point Hamilton Depression Rating Scale (HDRS). Secondary endpoints included a change on the Quick Inventory of Depressive Symptoms Self Report (QIDS-SR) scale. At baseline, the Brief Psychiatric Rating Scale (BPRS) was also administered. Psychiatric safety endpoints were assessed via standard scales for psychosis and the Columbia suicide scale. Other safety endpoints included cardiovascular, respiratory and central nervous system adverse events determined by hemodynamic and respiratory monitoring. Nitrous oxide-induced inactivation of vitamin B₁₂ was determined by measurement of plasma total homocysteine before and after treatment.

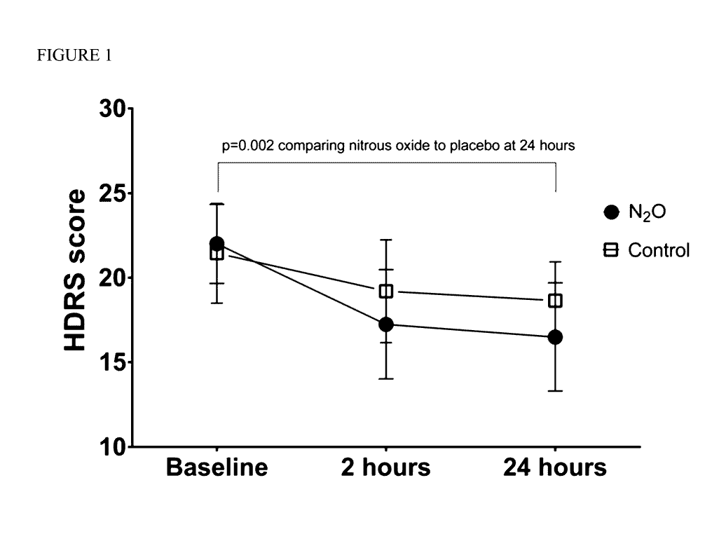

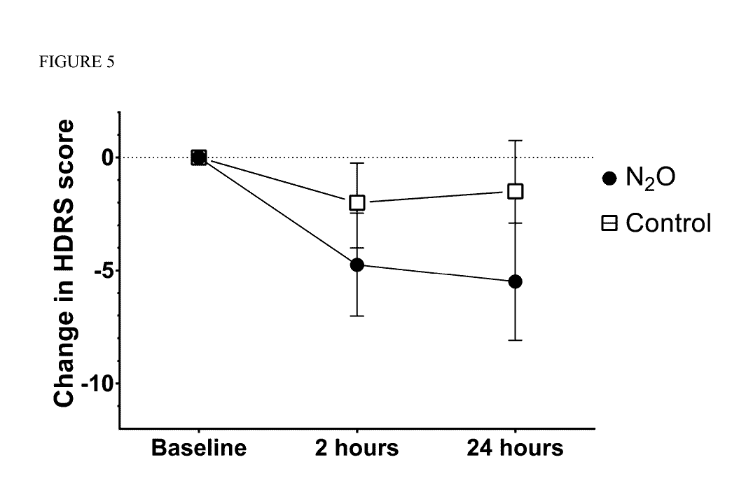

Patients experienced a significant improvement in depressive symptoms at 24 hours after receiving nitrous oxide compared to placebo (mean change in HDRS score −5.5, 95% CI −2.5 to −8.5 points, p=0.002 compared to baseline and placebo; FIG. 1; FIG. 5 shows the relative change adjusted for baseline). Supplemental Figure S2 shows the response on the self-reported QIDS scale.

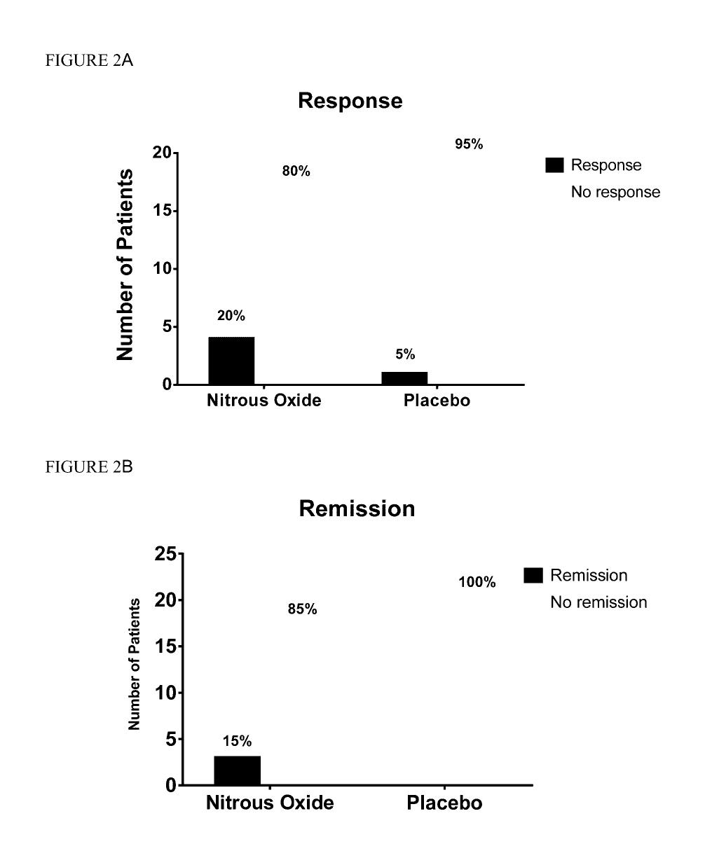

At 24 hours, four patients (20%) had treatment response (defined as reduction in depressive symptoms >50% on the HRDS) after receiving nitrous oxide compared to one patient (5%) after placebo treatment (odds ratio [OR] 4.0, 95% CI 0.45-35.79; FIG. 2A). Three patients (15%) had a full remission after nitrous oxide treatment (defined as complete resolution of depressive symptoms, HRDS <7 points), and none after placebo (OR 3.0, 95% CI 0.31-28.8; FIG. 2B).

Using the five levels of depression severity on the HRDS (normal/mild/moderate/severe/very severe), 7 out of twenty patients (35%) had at least a 2-level improvement at 24 hours after receiving nitrous oxide, i.e. from severe to mild, compared to two patients receiving placebo (10%; p=0.06; Table 3). Table 4 shows the response on the QIDS scale.

TABLE 3
Change in Level of Depression Severity 24 hours after Treatment

| | Nitrous Oxide | | | Control | | |
|---|---|---|---|---|---|---|
| Worse | [symbol] 1 | 0 | | [symbol] 1 | 1/20 | (5%) |
| Neutral | 0 | 6/20 | (30%) | 0 | 12/20 | (60%) |
| Better | [symbol] 1 | 7/20 | (35%) | [symbol] 1 | 5/20 | (25%) |
| | [symbol] 2 | 3/20 | (15%) | [symbol] 2 | 1/20 | (5%) |
| | [symbol] 3 | 3/20 | (15%) | [symbol] 3 | 1/20 | (5%) |

| | | | | |
|---|---|---|---|---|
| [symbol] 4 | 1/20 | (5%) | [symbol] 4 | 0 |

TABLE 4

| Nitrous Oxide | | Control | |
|---|---|---|---|
| [symbol] 1 | 2/20 (10%) | [symbol] 1 | 3/20 (15%) |
| 0 | 10/20 (50%) | 0 | 12/20 (60%) |
| [symbol] 1 | 3/20 (15%) | [symbol] 1 | 5/20 (25%) |
| [symbol] 2 | 5/20 (25%) | [symbol] 2 | 0 |

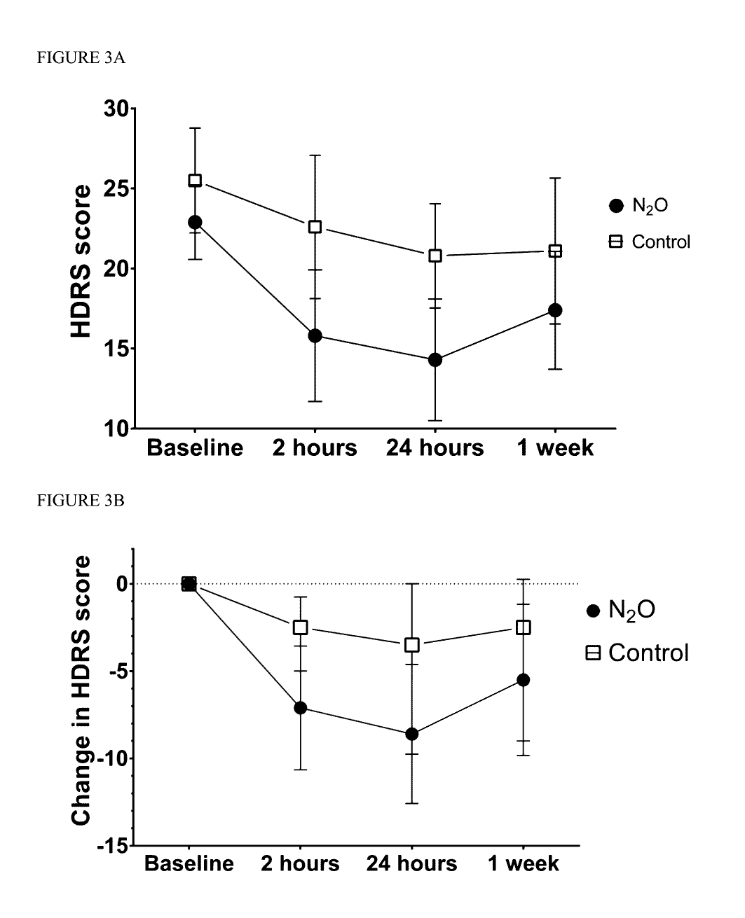

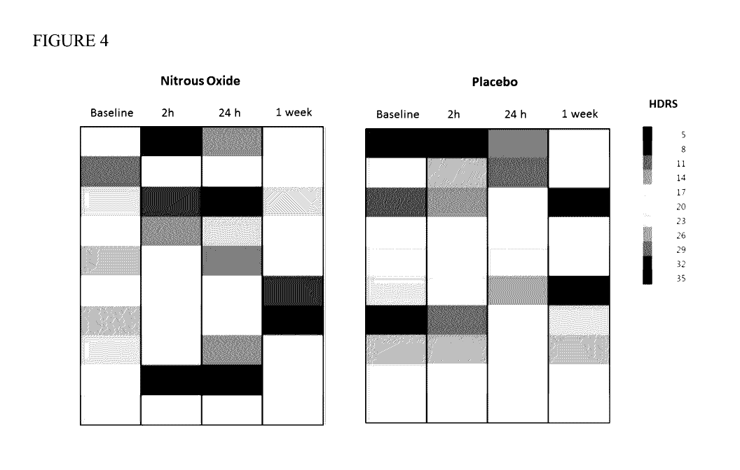

In this crossover trial, we expected depressive symptoms to revert to baseline when patients returned for their second treatment after one week. However, several patients showed markedly lower HRDS scores after one week. To address this carryover effect, we performed two additional analyses. First, we analyzed only the first treatment session, i.e., compared the 10 patients who received nitrous oxide to 10 who received placebo, akin to a parallel group design. Compared to patients who had placebo as first treatment, patients who received nitrous oxide first had a significant improvement of their depressive symptoms at 24 hours (mean reduction of HRDS −8.6 points, 95% CI −4.4 to −12.8 compared to −0.9 points, 95% CI 3.3 to −5.1 for placebo; FIG. 3A+B). The cell plot (heat map) in FIG. 4 shows individual HRDS scores at baseline before treatment, 2 hours, 24 hours and 1 week after treatment, indicative of a carryover and sustained treatment effect of nitrous oxide. Second, we included a 3-way interaction term in linear mixed model (treatment; time; and randomization group) to adjust for the carryover effect.

### Statistical Analysis

The primary outcome was analyzed with a mixed effects repeated measures linear model that included three interactions (treatment; time; randomization group) using restricted maximum likelihood estimation. Because of the observed carryover effect, two separate analyses were performed; one for the full cohort and one that only included the first treatment.

For the analysis of paired data, such as the levels of HRDS, we used the Wilcoxon signed-rank test. To compare the rates of treatment responses and remissions between the two treatments and using the paired data structure, an exact binomial test was used (and corresponding odds ratios calculated) as the number of discordant pairs was <20. Data are presented as mean±SD or 95% confidence intervals, or as median and interquartile range. Because this is the first in-human patient pilot study, no prior knowledge existed for adequate sample size determination. We based our sample size (20 treatment-resistant patients and 20 non-treatment resistant patients) on the available results from the ketamine trials, where a significant effect was observed in less than 20 patients. JMP Pro 11.1 and SAS 9.3 (SAS Institute, Cary, N.C.), as well as Prism 6.04 (GraphPad Software, Inc., La Jolla, Calif.) were used for the statistical analysis and graphing.

### Results:

The mean duration of nitrous oxide treatment was 55.6±2.5 (SD) minutes at an average inspiratory nitrous oxide concentration of 44% (37-45%, median, IQR). In five patients, the nitrous oxide treatment had to be discontinued due to adverse events (none during placebo). Patients experienced a significant improvement in depressive symptoms at 24 hours after receiving nitrous oxide compared to placebo (mean change in HDRS score −5.5, 95% CI −2.5 to −8.5 points, p=0.002). Four patients (20%) had treatment response (defined as reduction in depressive symptoms >50% on HRDS) with nitrous oxide compared to one patient (5%) after placebo (odds ratio [OR] 4.0, 95% CI 0.45-35.79). Three patients (15%) had a full remission after nitrous oxide treatment (defined as complete resolution of depressive symptoms, HRDS <7 points), and none after placebo (OR 3.0, 95% CI 0.31-28.8). No serious adverse event occurred. All adverse events were temporary and resolved shortly after completion.

### Conclusions:

This proof-of-concept trial showed that nitrous oxide has rapid and marked antidepressant effects in patients with treatment-resistant depression.

Compared to ketamine, the most commonly investigated NMDA receptor antagonist drug in major depressive disorder, nitrous oxide had a similarly rapid onset of antidepressant action (within two hours), but appeared to be devoid of psychotomimetic side effects seen with ketamine (delusion, hallucination). The fact that both ketamine and nitrous oxide share comparable antidepressant effects in patients with treatment-resistant depression, supports the notion that NMDA receptor signaling plays a crucial role in the neurobiology of major depressive disorder.

Although the euphoric effects of nitrous oxide have been known since the days of the “laughing gas parties” in the 1790's and although the drug continues to be recreationally used to this day, to our knowledge there has been no formal investigation about the antidepressant effects of nitrous oxide in patients with major depressive disorder. This proof-of-concept trial showed that nitrous oxide has rapid and marked antidepressant effects in patients with treatment-resistant depression. The antidepressant effects after a single, one-hour treatment with 50% nitrous oxide were sustained for at least 24 hours and in some patients even for 1 week. Nitrous oxide caused a treatment response in 20% of patients and a full remission in 15%. Furthermore, the lack of severe adverse events and mild to moderate nature of observed adverse events suggest an acceptable risk/benefit ratio for nitrous oxide use in the setting of major depressive disorder.

The internal validity of our crossover trial was affected by the observed carryover effect (patients having a different baseline at different treatment sessions). Typically, carryover effects bias results towards the null hypothesis, i.e., reduce the observable effect size. This was the case in our study: the ten patients who received the nitrous oxide treatment first had a mean reduction in depressive symptoms of 8.6 points on HRDS compared to 5.5 points for the full cohort. This observation supports the notion that the antidepressant effects of nitrous oxide are real. A second effect that influenced the internal validity of our trial was the presence of a placebo effect. Placebo effects are common in trials of antidepressants and may introduce bias by masking or exaggerating treatment effects.

In summary, this proof-of-concept clinical trial provided evidence that nitrous oxide has rapid and marked antidepressant effects in patients with treatment-resistant depression.

### OTHER EMBODIMENTS

The detailed description set-forth above is provided to aid those skilled in the art in practicing the present disclosure. However, the disclosure described and claimed herein is not to be limited in scope by the specific embodiments herein disclosed because these embodiments are intended as illustration of several aspects of the disclosure. Any equivalent embodiments are intended to be within the scope of this disclosure. Indeed, various modifications of the disclosure in addition to those shown and described herein will become apparent to those skilled in the art from the foregoing description, which do not depart from the spirit or scope of the present inventive discovery. Such modifications are also intended to fall within the scope of the appended claims.

1. A method of treating a depressive disorder in a subject in need thereof, comprising administering to the subject an effective amount of an inhaled gas comprising nitrous oxide.

2. The method according to claim 1, wherein the depressive disorder is atypical depression, bipolar disorder, catatonic depression, depressive disorder not otherwise specified, depressive personality disorder, double depression, dysthymia, major depressive disorder, melancholic depression, minor depressive disorder, postpartum depression, post-traumatic stress disorder, psychotic major depression, recurrent brief depression, seasonal affective disorder, suicidality/acute suicide risk, or treatment-resistant major depression.

3. The method according to claim 1, wherein the depressive disorder is treatment-resistant major depression.

4. (canceled)

5. The method according to claim 1, wherein the amount of nitrous oxide administered is sub-anesthetic.

6-7. (canceled)

8. The method according to claim 1, wherein the inhaled gas comprises from about 5% to about 70% nitrous oxide by weight.

9. The method according to claim 1, wherein the inhaled gas further comprises oxygen, nitrogen, xenon, or combinations thereof.

10. The method according to claim 9, wherein the inhaled gas comprises no more than 75% oxygen by weight.

11-14. (canceled)

15. The method according to claim 8, wherein the inhaled gas is administered at a flow rate of about 1.0 liters to about 2.0 liters per minute.

16. The method according to claim 1, wherein the inhaled gas is administered for about 1 to 90 minutes.

17. The method according to claim 1, wherein the inhaled gas is administered for about 30 to 60 minutes.

18. The method according to claim 1, wherein the inhaled gas is administered daily.

19. The method according to claim 1, wherein the inhaled gas is administered every other day.

20-23. (canceled)

24. The method according to claim 1, wherein the inhaled gas is administered weekly.

25-30. (canceled)

31. The method according to claim 1, further comprising the sequential or co-administration of an additional agent for the treatment and/or prevention of a functional Vitamin B₁₂ deficiency.

32. The method according to claim 1, wherein the additional therapeutic agent is Vitamin B₁₂, Vitamin B₆, Vitamin B₂, folic acid, methionine, cyanocobalamin, hydroxocobalamin, methylcobalamin, adenosylcobalamin, or combinations thereof.

33-35. (canceled)

36. An inhaled gas comprising nitrous oxide for use in treating a depressive disorder in a subject in need thereof.

37. Use of an inhaled gas comprising nitrous oxide for the manufacture of a medicament to treat a depressive disorder in a subject in need thereof.
